(19) United States
(12) Patent Application Publication
(10) Pub. No.: US 2015/0142038 A1
(43) Pub. Date: May 21, 2015

(54) STYLET CUTTING TIP FOR MEDICAL DEVICE, AND METHOD OF MAKING SAME

(71) Applicant: Cook Medical Technologies LLC, Bloomington, IN (US)

(72) Inventor: Jeffry Scott Melsheimer, Springville, IN (US)

(21) Appl. No.: 14/519,506

(22) Filed: Oct. 21, 2014

Related U.S. Application Data
(60) Provisional application No. 61/907,094, filed on Nov. 21, 2013.

(51) Int. Cl. A61B 17/34; B24B 3/60

(52) U.S. Cl. CPC A61B 17/3421; B24B 3/605; A61B 2017/3454; A61B 2017/00526
USPC 606/185; 451/58

(57) ABSTRACT

A medical device includes a housing, and a stylet within the housing having a cutting tip upon a stylet shaft. The cutting tip has a plurality of cutting edges in a regular distribution, each being formed by adjoining facets in an alternating arrangement with the cutting edges. The cutting edges are from six to sixteen in number and each slope toward the stylet center axis, such that the cutting tip forms a centrally located point. The facets each have a continuously concave curvature, such that the cutting edges each have a double hollow profile.

### TECHNICAL FIELD

The present disclosure relates generally to a stylet for a medical device, and more particularly to double hollow ground cutting edges on a stylet tip.

### BACKGROUND

During many medical procedures, it is necessary to push a cylindrical body through skin, fascia, muscle, fat or other membranes without “coring” the subject tissue. This is commonly facilitated by an indwelling sharp-tipped mechanism within the central lumen of the cylindrical body. Sharp tips on such mechanisms have been designed in a great many different ways, reaching back well over a century. So-called trocars, pencil-points, bevels and others have been the mainstay but typically exhibit shortcomings.

Pencil-points, where the sharpened tip of a device is generally conical in shape, tend to substantially deform tissue upon entry to the point that tears occur, resulting not only in a protracted healing process but also increased pain to the patient. Trocars, generally formed by intersecting faces in a pyramidal configuration can have more of a slicing effect as opposed to deformation and tearing. Innumerable different beveled and knife-edged designs have been proposed, and can slice tissues analogous to trocars. Conventional designs of all types could nevertheless be improved upon. This is true from the standpoint of manufacturability, as well as clinical efficacy, particularly with regard to the force required to penetrate into or through body tissues.

Commonly owned U.S. Pat. No. 6,450,973 to Murphy is directed to a kit of parts for taking a biopsy sample from a hard tissue. An interior shaft and an exterior sleeve form a tip for piercing the tissue. Murphy appears to be configured such that when a biopsy gun employing the needle is fired, recoil is advantageously absorbed by the shaft. Murphy also appears to utilize a sharp tipped stylet having faces in a generally pyramidal configuration.

### SUMMARY OF THE DISCLOSURE

In one aspect, a stylet and cannula assembly for a medical device includes a cannula having an elongate cannula body defining a cannula center axis, and formed therein a central lumen extending between a proximal cannula body end and a distal cannula body end. A stylet including an elongate stylet shaft defining a stylet center axis is arranged coaxially with the cannula within the central lumen, and has a cutting tip upon the elongate stylet shaft. The cutting tip projects out of the distal cannula end and includes a plurality of cutting edges having a regular distribution about the stylet center axis, and each being formed by adjoining ones of a plurality of facets in an alternating arrangement with the plurality of cutting edges. The plurality of cutting edges are from six to sixteen in number and each slope toward the stylet center axis, such that the cutting tip forms a centrally located stylet point. The plurality of facets each have a continuously concave curvature, such that each of the plurality of cutting edges has a double hollow profile in an axial section plane.

In another aspect, a method of making a medical device includes supporting a stylet of the medical device upon a support mechanism in a grinding apparatus, and reciprocating a rotating grinding wheel of the grinding apparatus relative the support mechanism along a grinding path angled toward a center axis of the stylet, in a plurality of grinding passes. The method further includes contacting the rotating grinding wheel with the stylet such that the contact removes material from a tip of the stylet to form a facet in each of the plurality of grinding passes. The method further includes rotating the stylet about its center axis relative the rotating grinding wheel between each of the plurality of grinding passes, such that each one of the facets adjoins two other facets to form a plurality of cutting edges upon the tip. The method still further includes indexing the rotation of the stylet such that the plurality of cutting edges are from six to sixteen in number in a regular distribution about the stylet center axis, and each has a double hollow profile in an axial section plane.

In still another aspect, a medical device includes a housing, and a stylet at a fixed position and orientation within the housing. The stylet includes an elongate stylet shaft defining a stylet center axis, and a cutting tip upon the stylet shaft. The cutting tip includes a plurality of cutting edges having a regular distribution about the stylet center axis, and each being formed by adjoining ones of a plurality of facets in an alternating arrangement with the plurality of cutting edges. The plurality of cutting edges are from six to sixteen in number and each slope toward the stylet center axis, such that the cutting tip forms a centrally located stylet point. The plurality of facets each have a continuously concave curvature, such that each of the plurality of cutting edges has a double hollow profile in axial section plane.

### BRIEF DESCRIPTION OF THE DRAWINGS

FIG. 1 is a side diagrammatic view of a medical device, according to one embodiment;

FIG. 2 is a partially sectioned side diagrammatic view of a portion of the medical device of FIG. 1;

FIG. 3 is a perspective view of a stylet of the medical device of FIGS. 1 and 2;

FIG. 4 is a sectioned view taken along line 4-4 of FIG. 2;

FIG. 5 is a sectioned view analogous to FIG. 4, according to another embodiment;

FIG. 6 is a sectioned view analogous to FIG. 4, according to yet another embodiment;

FIG. 7 is a side diagrammatic view at one stage of making the medical device of FIG. 1; and

FIG. 8 is a partially sectioned view taken along line 8-8 of FIG. 7.

### DETAILED DESCRIPTION

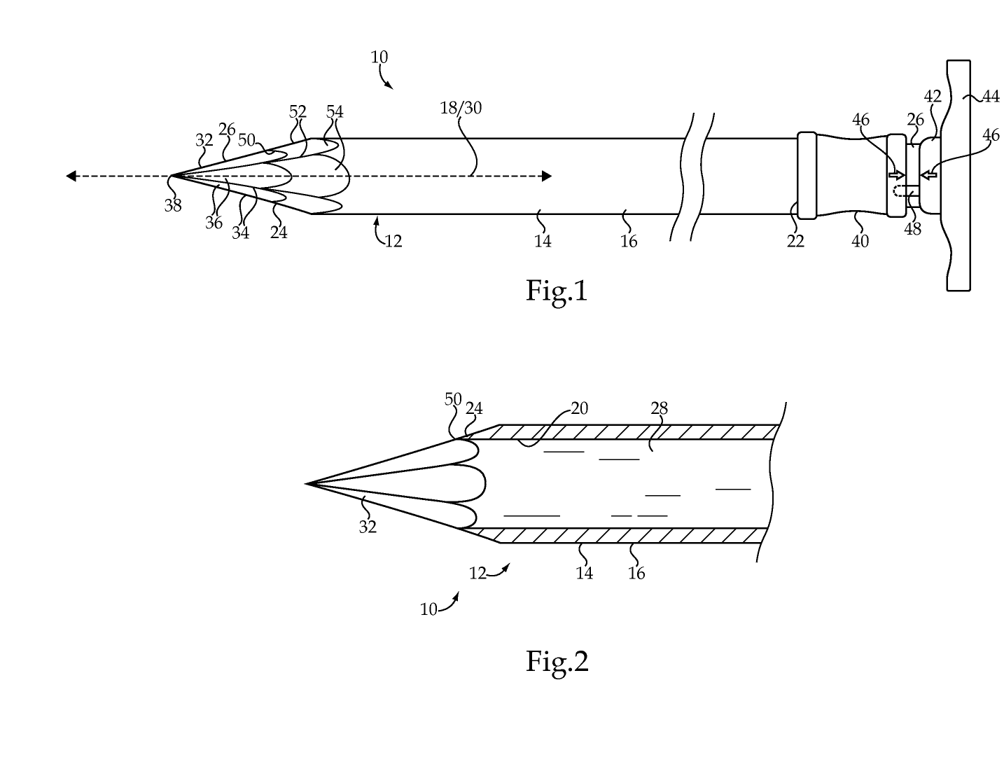

Referring to FIGS. 1 and 2, there is shown a medical device 10 according to one embodiment and including a stylet and medical device housing assembly 12, where the housing includes a cannula 14. Cannula 14 includes an elongate cannula body 16 defining a cannula center axis 18. Cannula body 16 has formed therein a central lumen 20, visible in the partially sectioned view of FIG. 2, which extends between a proximal cannula body end 22 and a distal cannula body end 24. Assembly 12 further includes a stylet 26 having an elongate stylet shaft 28 defining a stylet center axis 30 and arranged coaxially with cannula 14 within central lumen 20. Stylet 26 further includes a cutting tip 32 upon stylet shaft 28 and projecting out of distal cannula body end 24. Device 10 may further include a cannula hub 40 attached to proximal cannula body end 22, and a stylet hub 42 attached to stylet 26. Stylet 26 may be fitted relatively closely within cannula 14, such that relatively little clearance extends between the two. In a practical implementation strategy, device 10 may be configured such that stylet 26 is held at a fixed position within cannula 14 via a locking mechanism 48, of a generally conventional design and shown diagrammatically in FIG. 1. Mechanism 48 may be used to engage hubs 42 and 40, for example, and fixed to one of hubs 42 and 40 while being selectively engageable with the other of hubs 42 and 40. In FIG. 1, a set of alignment arrows 46 are provided on each of hubs 40 and 42 and can be used to indicate to a user that cannula 14 and stylet 6 are at a desired rotational orientation relative one another. In the illustrated embodiment, device 10 is shown as it might appear configured as a biopsy specimen obtaining device, where assembly 12 can be advanced into tissue in a patient of which a sample is desired. In general terms, stylet 26 and cannula 14 may be advanced together into the target tissue, and then stylet 26 pulled back to enable cannula 14 to be further advanced to obtain a core sample or the like from the target tissue.

To this end, cannula 14 may include a coring edge 50 having a scalloped configuration and extending circumferentially around cannula center axis 18. Cannula 14 may further include a plurality of cutting edges 52 having a regular distribution about center axis 18, and each being formed by adjoining ones of a plurality of facets 54 in an alternating arrangement with the plurality of cutting edges 52. In a practical implementation strategy, the cutting edges and facets upon distal cannula body end 24 may be shaped, distributed, and sized such that distal cannula body end 24 is congruent with cutting tip 32 of stylet 26. In other embodiments, the configuration of distal body end 24 might be not congruent with cutting tip 32, and stylet 26 might be used with a housing different from a cannula. Cutting tip 32 includes a plurality of cutting edges 34 having a regular distribution about stylet center axis 30. Each of cutting edges 34 are formed by adjoining ones of a plurality of facets 36 in an alternating arrangement with cutting edges 34. Cutting edges 34 may be from six to sixteen in number, and each slope toward stylet center axis 30, such that cutting tip 32 forms a centrally located stylet point 38, intersected by axis 30. Facets 36 each have a continuously concave curvature, as further discussed herein, such that each of cutting edges 34 has a double hollow profile in an axial section plane.

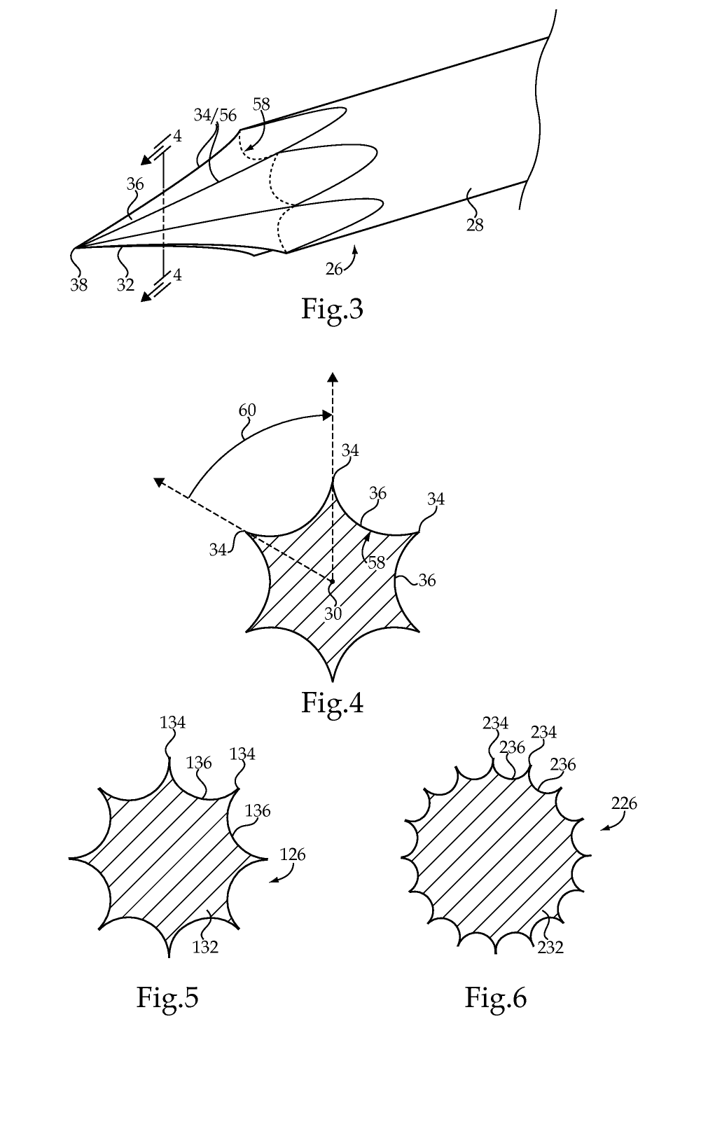

Referring also now to FIG. 3, there is shown a perspective view of stylet 26, illustrating the general convergence of cutting edges 34 and facets 36 toward point 38. In a practical implementation strategy, each of cutting edges 34 defines a perimetric edge line 56, and each of facets 36 extends from a corresponding first one of edge lines 56 to a corresponding second one of edge lines 56, according to the continuously concave curvature. As used herein, the term continuously concave should be understood to mean that at no point do facets 56 reverse curvature to a convex form. This characteristic may be further understood to mean that no true tangent line, intersecting a single point on any one of facets 56, can be drawn which is external to stylet 26. In one practical implementation strategy, the continuously concave curvature is uniform, and each of facets 36 defines a radius of curvature 58 which is uniform irrespective of what point along the curvature of each facet 36 between the corresponding edge lines 56 is being considered. As noted above, it is the continuously concave curvature of facets 36 that imparts a double hollow profile to cutting edges 34.

Referring also now to FIG. 4, there is shown a sectioned view taken along line 4-4 of FIG. 3, and illustrating each of six cutting edges 34 in an alternating arrangement with six facets 36. An included angle 60 between adjacent cutting edges 34 may be about 60° in the illustrated embodiment. Where a greater number of facets and cutting edges are used the included angle will of course be smaller. Radius 58 is also shown in FIG. 4, and it can be appreciated that radius 58 is uniform all the way across the corresponding facet 36. As such, each of facets 36 in the FIG. 4 embodiment has a profile which is generally that of a circular arc, although in other embodiments the profile might be parabolic or some other curvature. It will be appreciated that due to the sloping of cutting edges 34 and facets 36 towards point 38, the section plane through each facet 36 where the circularity of that facet's profile is apparent will be slightly different. In other words, circular arc segments in the profiles of facets 36 may not all be apparent in the same section plane, but are normalized in FIG. 4 and the other drawings for ease of illustration.

Referring to FIG. 5, there is shown a stylet 126 according to another embodiment, and having a cutting tip 132 with a plurality of cutting edges 134 in an alternating arrangement with a plurality of facets 136. Cutting edges 134 and facets 136 are eight in number, and it will thus be understood that included angles between cutting edges 134 are smaller than that of the FIG. 4 embodiment. Analogously, radiuses of curvature defined by facets 136 will be smaller than radius 58, at least for a similarly sized stylet. In FIG. 6 yet another embodiment of a stylet 226 is shown, having a cutting tip 232 with a plurality of cutting edges 234 in an alternating arrangement with a plurality of facets 236. In stylet 226, cutting edges 234 and facets 236 are sixteen in number.

As noted above, stylets according to the present disclosure may have cutting edges, as well as facets, from six to sixteen in number. FIGS. 4, 5 and 6 illustrate this general range. In practical implementation strategies, the number of cutting edges and number of facets may be from six to eight. In such embodiments, where the cutting edges are from six to eight in number but not necessarily strictly limited to such a range, each of the plurality of facets may define a radius R according to the equation:

$$\frac{\sqrt{C}}{\sqrt{( N - 1 )}} = R$$

where:
C=circumference of the stylet shaft; and
N=number of cutting edges.
   The above equation can be used as a guide in determining how to design a stylet according to the present disclosure. For example, for a stylet having a circumference of about C=0.1429 inches and a number of cutting edges N=6, each of the plurality of facets would define a radius R=0.170 inches. For a similar stylet having eight cutting edges, the radius may be about R=0.143 according to the above equation. For a stylet where circumference is about C=0.286 inches, having sixteen cutting edges, the radius may be about R=0.138 inches. Those skilled in the art will thus see how facet radius size changes in relation to cutting edge number and stylet circumference. In general terms, a greater number of cutting edges can be expected to have greater cutting efficacy, requiring less penetration force and deforming a patient's skin or other tissue relatively less upon stylet entry, causing a relatively lower level of patient discomfort, as compared with a lesser number. On the other hand, grinding a relatively greater number of facets/cutting edges is more labor and time intensive, particularly when considering the need to dress a grinding wheel relatively frequently. For manufacturing stylets contemplated herein having six to eight cutting edges, it may be necessary to dress a grinding wheel once every three stylets, in other words meaning that the grinding wheel is typically only capable of grinding somewhere from eighteen to twenty four facets before it needs to be dressed. The relationships set forth herein according to the above equation balance factors of manufacturability and cutting efficacy, reflecting the research insights into where a point of diminishing returns may occur respecting cutting edge number. Another way to understand these principles is that at some point, typically at about sixteen cutting edges but in particular from six to eight cutting edges, the additional benefits to cutting efficacy with a greater number of cutting edges can be outweighed by reduced manufacturability. These principles can be expected to scale with stylet size, applying not only to the above stylets where circumference is from about C=0.1429 inches to about C=0.286 inches, but also potentially to an even broader range.

### INDUSTRIAL APPLICABILITY

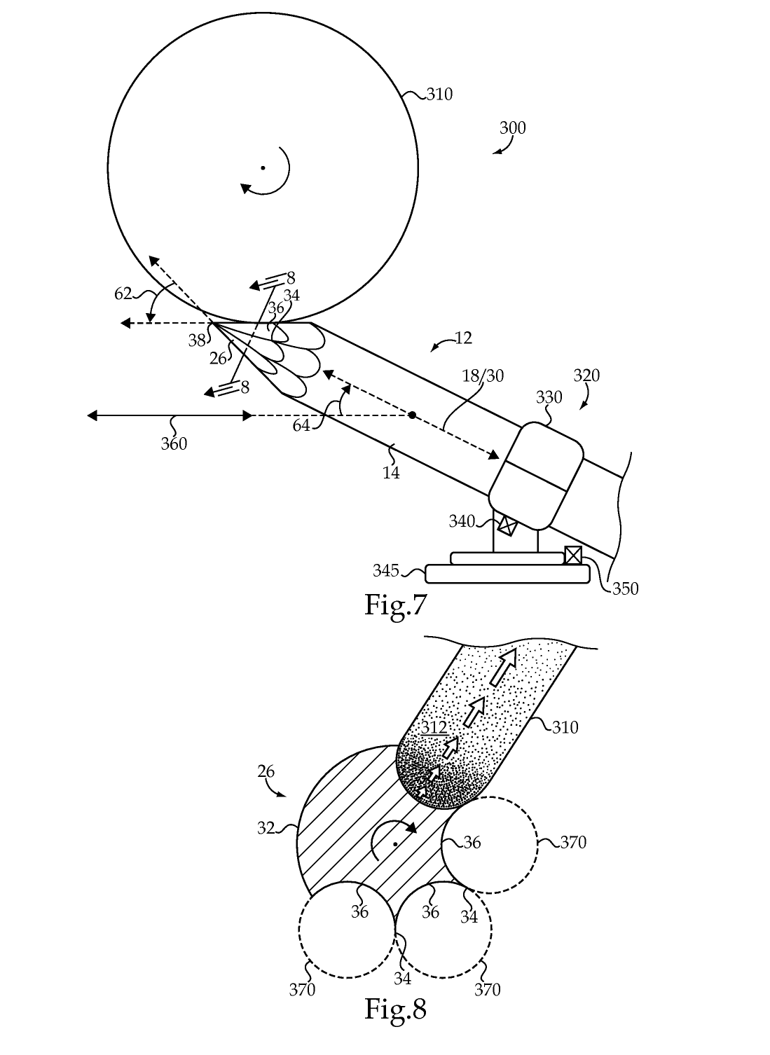

Referring to the drawings generally, but in particular now to FIG. 7, there is shown assembly 12 as it might appear at one stage of being made, according to the present disclosure. In FIG. 7, assembly 12 is positioned such that stylet 26 is supported upon a support mechanism 320 in a grinding apparatus 300. In the illustrated embodiment, assembly 12 is clamped within a collet 330 or another suitable supporting and clamping mechanism, which is coupled with a base 345. A linear actuator 350 which could be motorized or manually operated, is provided for adjusting mechanism 330 in linear directions. A rotary actuator 340 is provided for rotating mechanism 330 such that assembly 12, including stylet 26, rotates about its axis 30. A rotating grinding wheel 310 is shown tangentially contacting stylet 26 and cannula 14. It will be noted that stylet 26 and cannula 14 are held in a fixed configuration, similar to the configuration shown for assembled medical device 10 in FIG. 1. Hubs 40 and 42 and locking mechanism 48 might be used at the processing stage of FIG. 7 to hold assembly 12 thusly.

In FIG. 7, three facets 36 have been ground on stylet 26, and also on cannula 14, via reciprocating rotating grinding wheel 310 relative support mechanism 320 along a grinding path. Grinding wheel 310 may be reciprocated back and forth in a linear grinding path relative mechanism 320, with line 360 representing the grinding path and angled toward center axis 30 of stylet 26. Grinding wheel 310, or more typically apparatus 320, may be reciprocated in this general manner in a plurality of grinding passes, with grinding wheel 310 contacting stylet 26, and typically also contacting cannula 14 in each of the plurality of grinding passes, such that the contact removes material from the tip of stylet 26 to form a facet in each of the grinding passes. It will thus be appreciated that the cutting edges and facets on cannula 14 will be ground at the same time as those of stylet 26.

Upon grinding each facet in stylet 26, and between grinding passes, stylet 26 may be rotated about its axis 30 such that each one of facets 36 adjoins two other facets to form the plurality of cutting edges upon tip 32 of stylet 26. An included angle 62 having as its vertex stylet point 38 is defined by tip 32, and may be from about 20° to about 24°. As used herein, the term “about” may be understood in the context of conventional rounding to a consistent number of significant digits. Accordingly, “about 20” means from 9.5 to 20.4, and so on. The rotation of stylet 26 may be indexed such that cutting edges 26 are from six to sixteen in number in a regular distribution about center axis 30, and each having a double hollow profile. An included angle 64 between grinding path 360 and axis 30 may be from about 10° to about 12°, in a practical implementation strategy. For a stylet with six cutting edges, the indexed rotation of stylet 26 may be about 60°. For sixteen cutting edges, the indexed rotation may be about 23°. It will therefore be understood that stylet 26, typically while positioned at a rotationally and axially fixed position within cannula 14 in assembly 12, is incrementally rotated a plurality of times until the desired number of both facets and cutting edges have been ground.

Referring also now to FIG. 8, there is shown a sectioned view along line 8-8 of FIG. 7. It will be appreciated that the sectioned view in FIG. 8 is mildly distorted from a true axial section view in a manner similar to that explained above with respect to FIG. 4. From FIG. 8 it can be seen that grinding wheel 310 is rotating in contact with stylet 26, in particular tip 32 to remove material, and is shown as it might appear approximately where a new facet has just been formed. Three other facets 36 and two cutting edges 34 have already been formed, and stylet 26 has been rotated an indexed amount between the formation of each of facets 36. From the state shown in FIG. 8, stylet 26 will be further rotated between the grinding pass which completes formation of the latest facet, and a subsequent grinding pass commencing formation of the next facet. In FIG. 8, imaginary circles 370 are shown which are defined by already formed facets 36. It may be noted that these circles overlap slightly at cutting edges 34. Circles 370 are shown in the plane of the page in FIG. 8, although it will be appreciated that such circles as are defined by circular arc segments of facets 36 would typically lie slightly out of the plane of the page in FIG. 8 but are shown within it for convenience. It can also be noted that an outer surface 312 of grinding wheel 310 is rotating in contact with stylet 26, and in particular an arcuate non-axial profile of surface 312 defines a uniform radius of curvature. The removal of material via the contact imparts a complementary uniform radius of curvature to each of facets 36. The overlap between imaginary circles 370 to form the double hollow profiles of cutting edges 34 will of course vary depending upon the axial position along stylet 26 being considered. In at least some embodiments, the extent of overlap between circles 370 will increase in a proximal to distal direction as facets 36 approach point 38. With all the facets and cutting edges ground, stylet 26 and cannula 14 will typically be electro-polished, sterilized, packaged and distributed for service as a biopsy device or in any other advantageous medical device application.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims.

What is claimed is:

1. A stylet and cannula assembly for a medical device comprising:
a cannula including an elongate cannula body defining a cannula center axis, and having formed therein a central lumen extending between a proximal cannula body end and a distal cannula body end;
a stylet including an elongate stylet shaft defining a stylet center axis and arranged coaxially with the cannula within the central lumen, and a cutting tip upon the elongate stylet shaft;
the cutting tip projecting out of the distal cannula end and including a plurality of cutting edges having a regular distribution about the stylet center axis, and each being formed by adjoining ones of a plurality of facets in an alternating arrangement with the plurality of cutting edges;
the plurality of cutting edges being from six to sixteen in number and each sloping toward the stylet center axis, such that the cutting tip forms a centrally located stylet point; and
the plurality of facets each having a continuously concave curvature, such that each of the plurality of cutting edges has a double hollow profile in an axial section plane.

2. The assembly of claim 1 wherein each of the plurality of cutting edges defines a perimetric edge line, and each of the plurality of facets extends from a corresponding first one of the perimetric edge lines to a corresponding second one, according to the continuously concave curvature.

3. The assembly of claim 2 wherein an included angle defined by the cutting tip and having the stylet point at its vertex is from about 20° to about 24°.

4. The assembly of claim 1 wherein the continuously concave curvature is uniform.

5. The assembly of claim 4 wherein the plurality of cutting edges are from six to eight in number, and each of the plurality of facets defines a radius R according to the equation:
$$\frac{\sqrt{C}}{\sqrt{( N - 1 )}} = R$$
where:
C=circumference of the stylet shaft; and
N=number of cutting edges.

6. The assembly of claim 1 wherein the distal cannula end has another plurality of cutting edges having a regular distribution about the cannula center axis, and each being formed by adjoining ones of another plurality of facets in an alternating arrangement with the another plurality of cutting edges.

7. The assembly of claim 6 wherein the distal cannula end is congruent with the cutting tip of the stylet.

8. The assembly of claim 6 wherein the cannula further includes a coring edge located on the distal cannula end having a scalloped configuration and extending circumferentially around the cannula center axis.

9. A method of making a medical device comprising the steps of:
supporting a stylet of the medical device upon a support mechanism in a grinding apparatus;
reciprocating a rotating grinding wheel of the grinding apparatus relative the support mechanism along a grinding path angled toward a center axis of the stylet, in a plurality of grinding passes;
contacting the rotating grinding wheel with the stylet such that the contact removes material from a tip of the stylet to form a facet in each of the plurality of grinding passes;
rotating the stylet about its center axis relative the rotating grinding wheel between each of the plurality of grinding passes, such that each one of the facets adjoins two other facets to form a plurality of cutting edges upon the tip; and
indexing the rotation of the stylet such that the plurality of cutting edges are from six to sixteen in number in a regular distribution about the stylet center axis, and each has a double hollow profile in an axial section plane.

10. The method of claim 9 further comprising the steps of positioning the stylet at a rotationally and axially fixed position within a cannula of the medical device to form an assembly therewith.

11. The method of claim 10 wherein the step of supporting further includes supporting the assembly of the stylet and the cannula.

12. The method of claim 9 wherein the step of reciprocating further includes reciprocating the rotating grinding wheel along a grinding path oriented at an angle from about 10° to about 12° from the center axis of the stylet.

13. The method of claim 12 wherein the step of contacting further includes contacting an outer surface of the rotating grinding wheel having an arcuate non-axial profile defining a uniform radius of curvature with the stylet, such that the removal of material imparts a complementary uniform radius of curvature to each of the facets.

14. The method of claim 13 wherein the step of indexing further includes indexing the rotation such that the double hollow profile of each of the plurality of cutting edges is formed by intersecting circular arc segments in profiles of adjoining facets.

15. The method of claim 14 wherein the step of indexing further includes indexing the rotation such that the plurality of cutting edges are from six to eight in number.

16. A medical device comprising:
a housing;
a stylet at a fixed position and orientation within the housing, and including an elongate stylet shaft defining a stylet center axis, and a cutting tip upon the stylet shaft;
the cutting tip including a plurality of cutting edges having a regular distribution about the stylet center axis, and each being formed by adjoining ones of a plurality of facets in an alternating arrangement with the plurality of cutting edges;
the plurality of cutting edges being from six to sixteen in number and each sloping toward the stylet center axis, such that the cutting tip forms a centrally located stylet point; and
the plurality of facets each having a continuously concave curvature, such that each of the plurality of cutting edges has a double hollow profile in an axial section plane.

17. The medical device of claim 16 further comprising a locking mechanism locking the stylet at the fixed position and orientation within the housing, and the locking mechanism being adjustable to an unlocked state for sliding or rotating the stylet relative the housing.

18. The medical device of claim 16 wherein the housing includes a cannula and the stylet is within the cannula, and wherein the cannula includes another plurality of cutting edges and another plurality of facets, congruent with the plurality of cutting edges and the plurality of facets of the stylet, and a coring edge extending circumferentially around the stylet and having a scalloped configuration.

19. The medical device of claim 17 wherein each of the plurality of facets defines a radius of curvature R according to the equation:
$$\frac{\sqrt{C}}{\sqrt{( N - 1 )}} = R$$
where:
C=circumference of the stylet shaft; and
N=number of cutting edges.
